(19) United States
(12) Patent Application Publication
(10) Pub. No.: US 2014/0302146 A1
(43) Pub. Date: Oct. 9, 2014

(54) Ophthalmic Aqueous Composition

(71) Applicants: Takahiro Kurose, Osaka-shi (JP); Takayuki Miyano, Osaka-shi (JP); Toru Okubo, Osaka-shi (JP); Mariyo Kato, Osaka-shi (JP)

(72) Inventors: Takahiro Kurose, Osaka-shi (JP); Takayuki Miyano, Osaka-shi (JP); Toru Okubo, Osaka-shi (JP); Mariyo Kato, Osaka-shi (JP)

(73) Assignee: ROHTO PHARMACEUTICAL CO.,LTD., Osaka (JP)

(21) Appl. No.: 14/130,709

(22) PCT Filed: Jul. 5, 2012
(86) PCT No.: PCT/JP2012/067193

§ 371 Date: Feb. 25, 2014

(30) Foreign Application Priority Data
Jul. 8, 2011 (JP) 2011-151652

(51) Int. Cl. A61K 9/107; A61K 31/122; A61K 31/125; A61K 31/045

(52) U.S. Cl. CPC A61K 9/107; A61K 31/045; A61K 31/122; A61K 31/125
USPC 424/489; 514/729; 514/690; 514/692; 514/739

(57) ABSTRACT

The present invention provides an ophthalmic aqueous composition having an oil-in-water emulsion including a vegetable oil, a non-ionic surfactant, and a terpenoid, wherein an average particle diameter of emulsion particles is within the range of 30 nm to 300 nm.

### TECHNICAL FIELD

The present invention relates to an ophthalmic aqueous composition.

### BACKGROUND ART

In recent years, dry eye patients who describe symptoms such as eye dryness and eye strain due to the abnormality of tears have been increased. Endogenous diseases such as Stevens-Johnson syndrome and Sjogren's syndrome, side effects of medicines that are continuously used for a long time, a decrease in the number of blinks because of VDT work by personal computers and the like, and a reduction in indoor humidity by the spread of air conditioners are suggested as the cause of dry eye. Furthermore, it is well known that dry eye symptoms tend to be increased among contact lens users.

As a method for treating dry eye, a method for replenishing insufficient tears by artificial tears from the outside is known. In addition, as other methods for treating dry eye, a method for suppressing drainage of tears from the lacrimal canaliculus by blocking the lacrimal punctum with a lacrimal punctum plug and a method for preventing dryness by using goggle-type glasses for dry eye are known.

However, these conventional methods do not sufficiently satisfy the effect of mitigating dry eye symptoms, and further improvement is desired.

On the other hand, blending of various components has been attempted so as to improve physical properties of an ophthalmic aqueous composition. For example, Patent Literature 1 discloses, as an ophthalmic aqueous composition whose viscosity is stabilized, a composition applied to a mucous membrane, containing a cellulose thickening agent and at least one vegetable oil selected from the group consisting of sesame oil, olive oil, soybean oil, peanut oil, almond oil, wheat germ oil, camellia oil, corn oil, rapeseed oil, sunflower oil, cottonseed oil, and palm oil.

### CITATION LIST

### Patent Literature

Patent Literature 1: Japanese Patent Application Laid-Open No. 2005-206598

### SUMMARY OF INVENTION

### Problems to be Solved by the Invention

The present invention was made in view of the above-described circumstances of the conventional art, and a main object thereof is to provide an ophthalmic aqueous composition having a preventive or therapeutic effect on dry eye. There is tear film breakup time (BUT) as one of important diagnostic criteria for dry eye, and, in particular, it is a subject of the present invention to provide a novel ophthalmic aqueous composition capable of effectively prolonging BUT. It is to be noted that BUT is an index calculated based on time until a part called a dry spot appears on the surface of a tear film by tear evaporation after a blink, and it is considered that longer BUT means that dryness of the eye is more suppressed.

### Means for Solving the Problems

In order to achieve the above-described object, the present inventors made extensive research. As a result, it was found that an aqueous composition comprising an oil-in-water emulsion simultaneously including specific three components, a vegetable oil, a non-ionic surfactant, and a terpenoid, wherein an average particle diameter of the emulsion particles is controlled to be within a specific range, completely beyond expectation, can improve stability of a tear film and prolong tear film breakup time (BUT), and furthermore, exert a protective effect for a corneal epithelial cell, and greatly mitigate dry eye symptoms by using this as eye drop and the like when it is dry. Moreover, the present inventors found that, the aqueous composition comprising the oil-in-water emulsion containing the above-described three components, and wherein the average particle diameter of emulsion particles is within the specific range, can exert the effect of suppressing inflammation of the eye surface caused by allergic substances, which is not known at all as effects of the respective components contained in the composition, is exerted. Furthermore, it was found that, since the aqueous composition having the above-described features has high antioxidative activity, the effect of relieving corneal epithelium disorder caused by oxidative stress can be expected. The present invention was achieved as a result of further research based on such new findings.

Accordingly, the present invention provides the following ophthalmic aqueous composition.

Item 1-1. An ophthalmic aqueous composition comprising an oil-in-water emulsion including a vegetable oil, a non-ionic surfactant, and a terpenoid, wherein an average particle diameter of emulsion particles is within the range of 30 nm to 300 nm.
Item 1-2. The composition according to the above item 1-1, wherein the vegetable oil is sesame oil.
Item 1-3. The composition according to the above item 1-1 or 1-2, wherein a content ratio of the vegetable oil in terms of the total amount of the vegetable oil is 0.001 to 5 w/v % based on the total amount of the ophthalmic aqueous composition.
Item 1-4. The composition according to any one of the above items 1-1 to 1-3, wherein the non-ionic surfactant is at least one selected from the group consisting of a polyoxyethylene sorbitan fatty acid ester, a polyoxyethylene hydrogenated castor oil, a polyoxyethylene castor oil, and a polyoxyethylene-polyoxypropylene block copolymer.
Item 1-5. The composition according to any one of the above items 1-1 to 1-4, wherein the non-ionic surfactant comprises a polyoxyethylene-polyoxypropylene block copolymer and another non-ionic surfactant.
Item 1-6. The composition according to the above item 1-5, wherein the non-ionic surfactant contains a polyoxyethylene-polyoxypropylene block copolymer and a polyoxyethylene hydrogenated castor oil.
Item 1-7. The composition according to any one of the above items 1-1 to 1-6, wherein a content ratio of the non-ionic surfactant is 0.001 to 5 w/v % based on the total amount of the ophthalmic aqueous composition.
Item 1-8. The composition according to the above item 1-7, wherein based on 1 part by mass of the total amount of the vegetable oil, 1 to 30 parts by mass of the non-ionic surfactant in terms of the total amount is contained.
Item 1-9. The composition according to any one of the above items 1-1 to 1-8, wherein the terpenoid is at least one selected from the group consisting of menthol, menthone, camphor, borneol, and geraniol.
Item 1-10. The composition according to the above item 1-9, wherein the terpenoid is menthol.
Item 1-11. The composition according to any one of the above items 1-1 to 1-10, wherein a content ratio of the terpenoid in terms of the total amount of the terpenoid is 0.0001 to 0.2 w/v % based on the total amount of the ophthalmic aqueous composition.
Item 1-12. The composition according to the above item 1-11, in which based on 1 part by weight of the total amount of the vegetable oil, 0.001 to 100 parts by weight of the terpenoid in terms of the total amount is contained.
Item 1-13. The composition according to any one of the above items 1-1 to 1-12, which is an eye drop or an eye wash.

Furthermore, the present invention provides a method for imparting the effect of stabilizing a tear film to the ophthalmic aqueous composition, a method for imparting a cornea protective effect to the ophthalmic aqueous composition, a method for imparting the effect of preventing or treating dry eye to the ophthalmic aqueous composition, a method for imparting the effect of preventing or treating allergic symptoms of the eye surface caused by allergic substances to the ophthalmic aqueous composition, a method for imparting the effect of preventing or treating ophthalmia (snow blindness) and the like caused by ultraviolet light or the like to the ophthalmic aqueous composition, or a method for imparting the effect of suppressing or relieving inflammation of the eye surface to the ophthalmic aqueous composition, which are described below.

Item 2-1. A method for imparting the effect of stabilizing a tear film to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.
Item 2-2. A method for imparting a cornea protective effect to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.
Item 2-3. A method for imparting the effect of preventing or treating dry eye to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.
Item 2-4. A method for imparting the effect of suppressing or relieving inflammation of an eye surface to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.
Item 2-5. A method for imparting the effect of preventing or treating allergic symptoms of an eye surface caused by allergic substances to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.
Item 2-6. A method for imparting the effect of preventing or treating ophthalmia (snow blindness) and the like caused by ultraviolet light or the like to an ophthalmic aqueous composition, which includes adjusting an average particle diameter of emulsion particles in an oil-in-water emulsion to a range of 30 nm to 300 nm by blending a vegetable oil, a non-ionic surfactant, and a terpenoid to the ophthalmic aqueous composition.

Furthermore, the present invention provides a method for stabilizing a tear film, a method for protecting a cornea, a method for preventing or treating dry eye, a method for preventing or treating allergic symptoms of the eye surface caused by allergic substances, a method for preventing or treating ophthalmia (snow blindness) and the like caused by ultraviolet light or the like, or a method for suppressing or relieving inflammation of the eye surface, which are described below.

Item 3-1. A method for stabilizing a tear film, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.
Item 3-2. A method for protecting a cornea, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.
Item 3-3. A method for preventing or treating dry eye, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.
Item 3-4. A method for suppressing or relieving inflammation of an eye surface, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.
Item 3-5. A method for preventing or treating allergic symptoms of an eye surface caused by allergic substances, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.
Item 3-6. A method for preventing or treating ophthalmia (snow blindness) and the like caused by ultraviolet light or the like, which includes bringing an ophthalmic aqueous composition comprising an oil-in-water emulsion including a vegetable oil, a non-ionic surfactant, and a terpenoid, and has an average particle diameter of emulsion particles in the range of 30 nm to 300 nm into contact with a cornea.

Furthermore, the present invention also provides the use of the aspect described below.

Item 4-1. Use of a vegetable oil, a non-ionic surfactant, and a terpenoid for producing an ophthalmic aqueous composition comprising an oil-in-water emulsion including a vegetable oil, a non-ionic surfactant, and a terpenoid, wherein an average particle diameter of emulsion particles in the range of 30 nm to 300 nm, and having the effect of preventing or treating dry eye, a cornea protective effect, the effect of preventing or treating allergic symptoms of an eye surface caused by allergic substances, the effect of preventing or treating ophthalmia (snow blindness) and the like caused by ultraviolet light or the like, or the effect of suppressing or relieving inflammation of an eye surface.

Furthermore, the present invention also provides the use of the aspect described below.

Item 19. Use of a composition comprising an oil-in-water emulsion including a vegetable oil, a non-ionic surfactant, and a terpenoid, and wherein an average particle diameter of emulsion particles in the range of 30 nm to 300 nm, as an ophthalmic aqueous composition having the effect of preventing or treating dry eye, a cornea protective effect, the effect of preventing or treating allergic symptoms of an eye surface caused by allergic substances, the effect of preventing or treating ophthalmia (snow blindness) and the like caused by ultraviolet light or the like, or the effect of suppressing or relieving inflammation of an eye surface.
Item 20. The use according to the item 19, in which the composition is the composition according to any one of the above items 1-1 to 1-13.

Furthermore, the present invention also provides the composition of the aspect described below.

Item 21. A composition comprising an oil-in-water emulsion including a vegetable oil, a non-ionic surfactant, and a terpenoid, and has an average particle diameter of emulsion particles in the range of 30 nm to 300 nm, for use as an ophthalmic aqueous composition having the effect of preventing or treating dry eye, a cornea protective effect, the effect of preventing or treating allergic symptoms of an eye surface caused by allergic substances, the effect of preventing or treating ophthalmia (snow blindness) and the like caused by ultraviolet light or the like, or the effect of suppressing or relieving inflammation of an eye surface.
Item 22. The composition according to the item 21, according to any one of the above items 1-1 to 1-13.

Furthermore, the present invention also provides the method for producing the ophthalmic aqueous composition of the aspect described below.

Item 23. A method for producing an ophthalmic aqueous composition having the effect of preventing or treating dry eye, a cornea protective effect, the effect of preventing or treating allergic symptoms of an eye surface caused by allergic substances, the effect of preventing or treating ophthalmia (snow blindness) and the like caused by ultraviolet light or the like, or the effect of suppressing or relieving inflammation of an eye surface, which includes forming an oil-in-water emulsion having an average particle diameter of emulsion particles in the range of 30 nm to 300 nm by addition of a vegetable oil, a non-ionic surfactant, and a terpenoid to a support containing water.
Item 24. The manufacturing method according to the item 23, wherein the ophthalmic aqueous composition is the composition according to any one of the above items 1-1 to 1-13.

### Advantageous Effects of Invention

The ophthalmic aqueous composition of the present invention can stabilize a tear film and prolong tear film breakup time (BUT) effectively. Moreover, the composition has the effect of protecting a cornea from dryness and is useful as a cornea protective agent. Based on these effects, the ophthalmic aqueous composition of the present invention can relieve dryness of the eye and exerts an exceptional effect on prevention or therapy of dry eye. Furthermore, the ophthalmic aqueous composition of the present invention is stable formulation, has few side effects, and has high usability as a cornea protective agent, a preventive or therapeutic agent for dry eye and the like. It is to be noted that, the above-described cornea protective effect suppresses breakup of a barrier function of the corneal epithelial cell as another effect, and thus, has the effect of suppressing exacerbation of allergic symptoms.

Furthermore, the ophthalmic aqueous composition of the present invention has the effect of suppressing RANTES production, and thus, also has the effect of suppressing inflammation of the eye surface caused by common allergic substances. Therefore, the composition of the invention of the present application is also useful as a preventive agent or a therapeutic agent for allergic symptoms such as inflammation of the eye surface caused by allergic substances.

Furthermore, the ophthalmic aqueous composition of the invention of the present application has high antioxidative activity, and thus, the effect of relieving corneal epithelium disorder caused by oxidative stress can be expected and the ophthalmic aqueous composition also has a cornea protective effect based thereon. Therefore, the composition of the invention of the present application is also useful as a preventive agent or a therapeutic agent for ophthalmia (snow blindness) and the like caused by ultraviolet light or the like based on the cornea protective effect.

### BRIEF DESCRIPTION OF DRAWINGS

FIG. 1 is a graph showing an evaluation result of the effect of improving tear stability obtained in Test Example 1.

FIG. 2 is a graph showing a calculation result of a cell viability obtained in Test Example 2.

FIG. 3 is a graph showing a quantitative result of a concentration of RANTES obtained in Test Example 4.

FIG. 4 is a graph showing a measurement result of antioxidant potential (Cu reduction power) obtained in Test Example 5.

### EMBODIMENTS FOR CARRYING OUT THE INVENTION

Hereinafter, an ophthalmic aqueous composition of the present invention will be described in detail.

It is to be noted that, in the present specification, a unit “%” of a content ratio or a blending ratio means “w/v %” and is synonymous with “g/100 mL”, unless otherwise specified.

In addition, in the present specification, the abbreviation “POE” means polyoxyethylene and the abbreviation “POP” means polyoxypropylene, unless otherwise specified.

1. Ophthalmic Aqueous Composition

The ophthalmic aqueous composition of the present invention contains a vegetable oil, a non-ionic surfactant, and a terpenoid, and is an oil-in-water emulsion having a specific average particle diameter. Hereinafter, specific contents of these respective components and the oil-in-water emulsion will be described in detail.

(1) Vegetable Oil

In the ophthalmic aqueous composition of the present invention, as the vegetable oil (referred to as just “(a) component” in some cases), oils derived from vegetables can be used without particular limitation. However, the vegetable oil defined in the present application excludes that corresponding to “(c) component” described below.

Examples of components contained in vegetable oils mainly include fatty acid triglyceride. Fatty acids constituting the fatty acid triglyceride are classified roughly into saturated fatty acids and unsaturated fatty acids, as unsaturated fatty acids, preferably contain one or more selected from the group consisting of medium-chain fatty acid triglyceride made from fatty acids having a carbon number of 8 to 24, such as palmitic acid, stearic acid, oleic acid, ricinoleic acid, linoleic acid, linolenic acid, behenic acid, lignoceric acid, icosanoic acid, myristic acid, and a palmitoleic acid, above all, more preferably contain one or more selected from the group consisting of linoleic acid and oleic acid, and further preferably contain both of linoleic acid and oleic acid. Furthermore, as the fatty acid composition of the vegetable oils, in the case of containing linoleic acid, 30 mass % or more, and preferably 40 mass % or more of linoleic acid is contained, and in the case of containing oleic acid, 20 mass % or more, and preferably 30 mass % or more of oleic acid is contained.

Regarding the vegetable oil blended into the ophthalmic aqueous composition of the present invention, a higher content ratio of the unsaturated fatty acids to the total of the fatty acids constituting the fatty acid triglyceride is preferable, and it is preferable that the content ratio of the unsaturated fatty acids to the total of the fatty acids be 50 mass % or more, more preferably 60 mass % or more, further preferably 70 mass % or more, and particularly preferably 80 mass % or more.

Examples of these vegetable oils include sesame oil, castor oil, soybean oil, peanut oil, almond oil, wheat germ oil, camellia oil, corn oil, rapeseed oil, sunflower oil, cottonseed oil and the like. These vegetable oils can be used singly or in a combination of two or more.

Among these vegetable oils, from the viewpoints that the effect of improving tear film stability and a cornea protective effect, which are the object of the present invention, are good, and further, from the viewpoints of the effect of suppressing RANTES and an antioxidation effect, for example, sesame oil can be suitably used.

Sesame oil is a vegetable oil obtained from seeds of a plant of the Sesamum genus of the Pedaliaceae family (Sesamumindicum Linne (Pedaliaceae) or the like). The sesame oil that can be blended into the ophthalmic aqueous composition of the present invention is not particularly limited as long as it is medicinally and pharmacologically (pharmaceutically) or physiologically acceptable. For example, an oil obtained from seeds using a known squeeze method and a known purification method or a commercially-available oil can be used. In particular, from the viewpoint of more remarkably exhibiting the effect of suppressing discoloration or turbidity over time of the ophthalmic aqueous composition, sesame oil that meets the standards of The Japanese Pharmacopoeia Fifteenth Edition is preferable.

The content ratio of the vegetable oil in the ophthalmic aqueous composition of the present invention can be appropriately set according to the kind of the vegetable oil, the kind and the content ratio of other blending components, a pharmaceutical formulation of the ophthalmic aqueous composition and the like. As one example of the content ratio of the vegetable oil, the total amount of the vegetable oil can be usually 0.001 to 5%, preferably 0.001 to 1%, and more preferably 0.001 to 0.5%, on the basis of the total amount of the ophthalmic aqueous composition. In particular, from the viewpoints of easily forming emulsion particles within an intended particle diameter range, improving tear film stability, making a cornea protective effect better, and improving formulation stability, about 0.001 to 0.1% is preferable.

(2) Non-Ionic Surfactant

The non-ionic surfactant (referred to as just “(b) component” in some cases) can be used without particular limitation as long as it is medicinally and pharmacologically (pharmaceutically) or physiologically acceptable non-ionic surfactant. For example, polyoxyethylene sorbitan fatty acid esters, polyoxyethylene hydrogenated castor oils, polyoxyethylene castor oils, polyoxyethylene-polyoxypropylene block copolymers, polyoxyethylene-polyoxypropylene block copolymer adducts, polyoxyethylene-polyoxypropylene alkyl ethers, polyoxyethylene alkyl phenyl ethers and the like can be used. These non-ionic surfactants may be used singly or in an arbitrary combination of two or more.

Specific examples of the above-described non-ionic surfactants include components as follows. Examples of the POE sorbitan fatty acid esters include POE (20) Sorbitan Monolaurate (Polysorbate 20), POE (20) Sorbitan Monooleate (Polysorbate 80), POE Sorbitan Monostearate (Polysorbate 60), and POE Sorbitan Tristearate (Polysorbate 65); examples of the POE hydrogenated castor oils include POE hydrogenated castor oil 5, POE hydrogenated castor oil 10, POE hydrogenated castor oil 20, POE hydrogenated castor oil 40, POE hydrogenated castor oil 50, POE hydrogenated castor oil 60, and POE hydrogenated castor oil 100; examples of the POE castor oils include POE castor oil 3, POE castor oil 10, POE castor oil 20, POE castor oil 35, POE castor oil 40, POE castor oil 50, and POE castor oil 60; examples of the polyoxyethylene-polyoxypropylene block copolymers (hereinafter, referred to also as “polyoxyethylene polyoxypropylene copolymer”) include Poloxamer 407 such as POE (196) POP (67) glycol; examples of the POE-POP block copolymer adducts of ethylenediamine include Poloxamine; examples of the POE alkyl ethers include POE (9) lauryl ether; examples of the POE•POP alkyl ethers include POE (20) POP (4) cetyl ether; and examples of the POE alkyl phenyl ethers include POE (10) nonylphenyl ether. It is to be noted that a figure in a parenthesis denotes the addition molar number.

Among these non-ionic surfactants, from the viewpoint of further improving the effects of the present invention, the POE sorbitan fatty acid esters, the POE hydrogenated castor oils, the POE castor oils, the polyoxyethylene-polyoxypropylene block copolymers and the like are preferable, and specifically, the POE hydrogenated castor oils, the POE castor oils, and the polyoxyethylene-polyoxypropylene block copolymers are preferable. Preferable specific examples of these respective non-ionic surfactants include Polysorbate 80, POE hydrogenated castor oil 60, POE castor oil 10, POE castor oil 35, and Poloxamer 407, and particularly preferably POE hydrogenated castor oil 60, POE castor oil 10, POE castor oil 35, and Poloxamer 407. It is more preferable that two or more of these non-ionic surfactants be used in combination. It is to be noted that, as polyoxyethylene hydrogenated castor oil 60, polyoxyethylene hydrogenated castor oil 60 that meets the standards of Japanese Pharmaceutical Excipients 2003 is preferable, and as polyoxyethylene castor oil 10 and polyoxyethylene castor oil 35, polyoxyethylene castor oils that meet the standards of Japanese Pharmaceutical Excipients 2003 are preferable.

In the ophthalmic aqueous composition of the present invention, from the viewpoint of further improving the effects of the present invention, it is particularly preferable that a combination of the polyoxyethylene-polyoxypropylene block copolymers and other non-ionic surfactants is used. In this case, as other non-ionic surfactants that are used by being combined with the polyoxyethylene-polyoxypropylene block copolymers, the POE hydrogenated castor oils, the POE castor oils and the like are preferable, and the POE hydrogenated castor oils are particularly preferable. As the specific combination, the combination of Poloxamer 407 and polyoxyethylene hydrogenated castor oil 60, the combination of Poloxamer 407 and POE castor oil 10, and the combination of Poloxamer 407 and POE castor oil 35 are preferable, and the combination of Poloxamer 407 and polyoxyethylene hydrogenated castor oil 60 is particularly preferable.

The content ratio of the non-ionic surfactant in the ophthalmic aqueous composition can be appropriately set according to the kind of the non-ionic surfactant, the kind and the content ratio of other blending components, a pharmaceutical formulation of the ophthalmic aqueous composition and the like. For example, from the viewpoints of further improving the effects of the present invention and making eye irritation milder, the total amount of the non-ionic surfactant should be usually 0.001 to 5%, preferably 0.001 to 1.5%, more preferably 0.001 to 1%, further preferably 0.005 to 1%, further more preferably 0.005 to 0.8%, and particularly preferably 0.01 to 0.7%, based on the total amount of the ophthalmic aqueous composition.

When the polyoxyethylene-polyoxypropylene block copolymers and other non-ionic surfactants are combined to be used as the non-ionic surfactant, it is preferable that the content ratio of the polyoxyethylene-polyoxypropylene block copolymers to other non-ionic surfactants be within the range of, based on 1 part by mass of the total amount of the polyoxyethylene-polyoxypropylene block copolymers, about 0.1 to 50 parts by mass, preferably about 0.2 to 20 parts by mass, more preferably about 0.5 to 10 parts by mass, further preferably about 0.6 to 5 parts by mass, and particularly preferably about 1 to 5 parts by mass in terms of the total amount of other non-ionic surfactants.

Although the content ratio of the non-ionic surfactant to the vegetable oil is not particularly limited in the ophthalmic aqueous composition of the present invention, in order to form an emulsion having a predetermined particle diameter and further improve the effects of the present invention, it is preferable that the content ratio be within the range of, based on 1 part by mass of the total amount of the vegetable oil, about 1 to 30 parts by mass, preferably about 2 to 25 parts by mass, more preferably about 3 to 20 parts by mass, and particularly preferably about 4 to 15 parts by mass in terms of the total amount of the non-ionic surfactant.

(3) Terpenoid

The ophthalmic aqueous composition of the present invention contains terpenoid (referred to as just “(c) component” in some cases). The kind of the terpenoid is not particularly limited as long as it is medicinally and pharmacologically (pharmaceutically) or physiologically acceptable. For example, menthol, menthone, camphor, borneol, geraniol, cineol, citronellol, carvone, anethole, eugenol, limonene, linalool, linalyl acetate, and derivatives thereof can be used. These compounds may be any of d-isomer, l-isomer, and dl-isomer. Furthermore, in the present invention, essential oils containing the above-described compounds may be used as the terpenoid. Examples of these essential oils include eucalyptus oil, bergamot oil, peppermint oil, coolmint oil, spearmint oil, mentha oil, fennel oil, cinnamon oil, rose oil, and camphor oil. These terpenoids may be used singly or in an arbitrary combination of two or more thereof.

Among these terpenoids, from the viewpoint of further improving the effects of the present invention, menthol, menthone, camphor, borneol, geraniol and the like are preferable. Menthol and camphor are further preferable, and l-menthol, dl-menthol, d-camphor, and dl-camphor are particularly preferable. Most preferable terpenoids for exhibiting the effects of the present invention are menthol such as l-menthol and dl-menthol.

In the ophthalmic aqueous composition of the present invention, the content ratio of the terpenoid can be appropriately set according to the kind of the terpenoid, the kind and the content ratio of other blending components, a pharmaceutical form of the ophthalmic aqueous composition and the like. For example, as one example of the content ratio of the terpenoid, from the viewpoint of further improving the effects of the present invention, the total amount of the terpenoid is 0.0001 to 0.2 w/v %, preferably 0.0005 to 0.1 w/v %, further preferably 0.0005 to 0.07 w/v %, further more preferably 0.001 to 0.07 w/v %, and particularly preferably 0.005 to 0.07 w/v %, on the basis of the total amount of the ophthalmic aqueous composition. It is to be noted that, in the case of using essential oils containing terpenoids, the terpenoid content in the essential oils to be blended should be set so as to satisfy the above-described content ratio.

Although the content ratio of the vegetable oil and the terpenoid described above is not particularly limited as long as it satisfies the content ratio of the above-described respective components, from the viewpoint of further improving the effects of the present invention, it is preferable that the ratio be, based on 1 part by weight of the total amount of the vegetable oil, 0.001 to 100 parts by weight, preferably 0.01 to 20 parts by weight, and further preferably 0.1 to 10 parts by weight in terms of the total amount of the terpenoid. It is to be noted that, in the case of using essential oils containing terpenoids, the terpenoid content in the essential oils to be blended should be set so as to satisfy the above-described ratio.

(4) Oil-in-Water Emulsion

The ophthalmic aqueous composition of the present invention is an oil-in-water emulsion including the above-described vegetable oil, non-ionic surfactant, and terpenoid as essential components.

The water content in the ophthalmic aqueous composition of the present invention should be usually 80 w/v % or more, more preferably 90 w/v % or more, further preferably 95 w/v % or more, and particularly preferably 97 w/v % or more. The water contained in the ophthalmic aqueous composition of the present invention may be water that is medicinally and pharmacologically (pharmaceutically) or physiologically acceptable. For example, distilled water, common water, purified water, sterile purified water, water for injection, distilled water for injection and the like can be used. These definitions are based on The Japanese Pharmacopoeia Fifteenth Edition.

The ophthalmic aqueous composition of the present invention comprises an oil-in-water emulsion including the above-described vegetable oil, non-ionic surfactant, and terpenoid as essential components, and the average particle diameter of emulsion particles is within the range of 30 nm to 300 nm. In particular, the average particle diameter of emulsion particles is preferably within the range of 30 to 270 nm, more preferably within the range of 31 to 270 nm, further preferably within the range of 35 to 265 nm, and particularly preferably within the range of 35 to 260 nm.

The condition that the above-described three components are contained and the condition that the average particle diameter of emulsion particles is within the range of 30 nm to 300 nm are simultaneously satisfied so that the effects such as improvement of tear film stability and protection of a cornea from dryness are remarkably exhibited to exert an excellent effect of mitigating dry eye symptoms, and at the same time, high formulation stability is obtained. The composition including the above-described three components and the average particle diameter of emulsion particles within the above-described range make these effects especially excellent, and a remarkable effect of mitigating dry eye symptoms and a remarkable formulation stability are obtained.

The above-described essential components and, if necessary, arbitrary components described below are added into water to be sufficiently mixed, and the oil-in-water emulsion is adjusted such that the average particle diameter of emulsion particles is within a predetermined range so that the ophthalmic aqueous composition of the present invention can be obtained.

It is to be noted that, in the present specification, the average particle diameter of emulsion particles is a value measured in accordance with conditions specified in Test Example 1 specified below using a particle diameter measuring device by dynamic light scattering.

(5) Other Components

The ophthalmic aqueous composition of the present invention needs to satisfy the condition that the above-described (a) to (c) components are contained and the average particle diameter of emulsion particles is within a predetermined range, and other components can be contained if necessary, as long as the condition is satisfied.

For example, in addition to the above-described (a) to (c) components, the ophthalmic aqueous composition of the present invention can further contain biguanide bactericides.

The biguanide bactericides are known bactericides as a monomer having at least one biguanide group [—NHC(═NH)NHC(═NH)NH—], a polymer composed of the monomer, and a salt form thereof, and may be manufactured by a known method or can be obtained as a commercial product.

The biguanide bactericides used for the present invention is not particularly limited as long as it is medicinally and pharmacologically (pharmaceutically) or physiologically acceptable, and examples thereof include at least one selected from the group consisting of polyhexanide and a salt thereof. Polyhexanide is referred to also as polyhexamethylene biguanide or PWVIB. By a combination of the above-described (a) to (c) components and the biguanide bactericides, the effects of the present invention can be further improved.

Although polyhexanide used for the present invention is not particularly limited as long as it is medicinally and pharmacologically (pharmaceutically) or physiologically acceptable, specific examples thereof include a compound represented by the following formula (1).

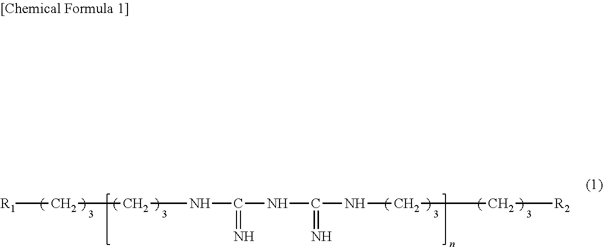

In the formula (1), R₁ and R₂ are the same or different and represent a group represented by the following formula (2) or an amino group. Preferably, R₁ is an amino group and R₂ is a group represented by the formula (2) or an amino group, and further preferably, R₁ is an amino group and R₂ is a group represented by the formula (2).

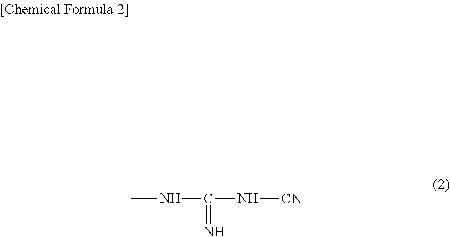

In addition, in the formula (1), n represent an integer of 1 to 500. An integer of 2 to 200 is preferable, an integer of 4 to 100 is further preferable, and an integer of 8 to 20 is particularly preferable.

The salt of polyhexanide is not particularly limited as long as it is medicinally and pharmacologically (pharmaceutically) or physiologically acceptable. Specific examples of the salt of polyhexanide include inorganic acid salts such as hydrochloride, hydrobromide, hydrosulfate, and borate; and organic acid salts such as acetate, gluconate, maleate, ascorbate, stearate, tartrate, and citrate. Among these salts of polyhexanide, inorganic acid salts are preferable, and hydrochloride is further preferable. One of these salts of polyhexanide may be used singly or in an arbitrary combination of two or more thereof.

In the ophthalmic aqueous composition of the present invention, the content ratio of the biguanide bactericide can be appropriately set according to the kind of the biguanide bactericide, the kind and the content ratio of other blending components, a pharmaceutical form of the ophthalmic aqueous composition and the like. For example, as one example of the content ratio of the biguanide bactericide, the total amount of the biguanide bactericide is in the range of 0.00001 to 0.01 w/v %, preferably 0.00002 to 0.001 w/v %, and further preferably 0.00004 to 0.0001 w/v %, on the basis of the total amount of the ophthalmic aqueous composition, from the viewpoint of further improving the effects of the present invention.

It is preferable that the ophthalmic aqueous composition of the present invention further contain boric acid or a salt thereof. Further improvement of the effects of the present invention is expected by containing boric acid or a salt thereof. As borate, alkali metal borate, alkali earth metal borate or the like can be used. In addition, as boric acid or a salt thereof, a borate hydrate can be used. More specific examples of boric acid or a salt thereof include boric acid, sodium borate, potassium tetraborate, potassium metaborate, ammonium borate, borax and the like. Boric acid or salts thereof can be used singly or in combinations of two or more. Preferable specific examples of boric acid or a salt thereof include the combination of boric acid and a salt thereof; preferably the combination of boric acid and an alkali metal slat and/or an alkali earth metal salt of boric acid; further preferably the combination of boric acid and an alkali metal salt of boric acid; and particularly preferably the combination of boric acid and borax.

In the case where boric acid or a salt thereof is contained in the ophthalmic aqueous composition of the present invention, the content ratio of boric acid or a salt thereof cannot be uniformly specified because it varies depending on the kind of boric acid or a salt thereof to be used, the kind and the amount of other blending components, use of the ophthalmic aqueous composition and the like, for example, on the basis of the total amount of the ophthalmic aqueous composition, the total amount of boric acid or a salt thereof may be 0.01 to 10 w/v %, preferably 0.05 to 5 w/v %, and further preferably 0.1 to 2 w/v %.

The ophthalmic aqueous composition of the present invention may further contain buffers. The buffers that can be blended into the ophthalmic aqueous composition are not particularly limited as long as they are medicinally and pharmacologically (pharmaceutically) or physiologically acceptable. One example of these buffers includes phosphate buffers, carbonate buffers, citrate buffers, acetate buffers, tris buffers, epsilon-aminocapronic acid, aspartic acid, and aspartate. These buffers may be combined to be used. Preferable buffers are phosphate buffers, carbonate buffers, and citrate buffers. Examples of the phosphate buffers include phosphoric acid, and phosphates such as alkali metal phosphate and alkali earth metal phosphate. Examples of the carbonate buffers include carbonic acid, and carbonates such as alkali metal carbonate and alkali earth metal carbonate. Examples of the citrate buffers include citric acid, alkali metal citrate, and alkali earth metal citrate. In addition, as the phosphate buffers, a phosphate hydrate may be used. More specific examples include phosphoric acid or a salt thereof (disodium hydrogenphosphate, sodium dihydrogenphosphate, potassium dihydrogenphosphate, trisodium phosphate, dipotassium phosphate, calcium monohydrogen phosphate, calcium dihydrogen phosphate or the like) as the phosphate buffers; carbonic acid or a salt thereof (sodium hydrogen carbonate, sodium carbonate, ammonium carbonate, potassium carbonate, calcium carbonate, potassium hydrogen carbonate, magnesium carbonate or the like) as the carbonate buffers; citric acid or a salt thereof (sodium citrate, potassium citrate, calcium citrate, sodium dihydrogencitrate, disodium citrate or the like) as the citrate buffers; acetic acid or a salt thereof (ammonium acetate, potassium acetate, calcium acetate, sodium acetate or the like) as the acetate buffers; tris(hydroxymethyl)aminomethane or a salt thereof (hydrochloride, acetate, sulfonate or the like) as the tris buffers; and aspartic acid or a salt thereof (sodium aspartate, magnesium aspartate, potassium aspartate or the like). One of these buffers may be used singly or in an arbitrary combination of two or more thereof.

Although pH of the ophthalmic aqueous composition of the present invention is not particularly limited as long as it is within the range that is medicinally and pharmacologically (pharmaceutically) or physiologically acceptable, from the viewpoints of more remarkably exhibiting the effect of suppressing discoloration or turbidity over time of the ophthalmic aqueous composition and remarkably exhibiting other effects of the invention of the present application, one preferable example of pH includes a range of 4 to 9.5, preferably 4.5 to 9, more preferably 4.5 to 8.5, and further preferably 4.5 to 8.

Furthermore, if necessary, the ophthalmic aqueous composition of the present invention can be adjusted to an osmotic pressure ratio within the range that is acceptable to biological bodies. The appropriate osmotic pressure ratio is, although it varies based on an application site, a dosage form or the like, usually within the range of 0.7 to 5, more preferably 0.8 to 3, and further preferably 0.9 to 2. The adjustment of the osmotic pressure can be performed by a known method in the art using inorganic salts, polyalcohols or the like. It is to be noted that, the osmotic pressure ratio here is a ratio of an osmotic pressure of a sample to the osmotic pressure of 286 mOsm (0.9 w/v % aqueous sodium chloride solution) based on The Japanese Pharmacopoeia Fifteenth Edition, and the osmotic pressure is a value measured in accordance with the osmotic pressure measuring method (freezing-point depression method) as described in The Japanese Pharmacopoeia Fifteenth Edition. It is to be noted that a standard solution for measuring an osmotic pressure ratio (0.9 w/v % aqueous sodium chloride solution) is prepared by drying sodium chloride (The Japanese Pharmacopoeia standard reagent) at 500 to 650° C. for 40 to 50 minutes, allowing it to cool in a desiccator (silica gel), precisely weighing out 0.900 g thereof, dissolving it in purified water, and precisely adjusting the amount to 100 mL, or a commercially available standard solution for measuring an osmotic pressure ratio (0.9 w/v % aqueous sodium chloride solution) is used.

In the ophthalmic aqueous composition of the present invention, within the range not impairing the effects of the present invention, in addition to the above-described components, various pharmacologically-active components or physiologically-active components are appropriately selected based on its use and pharmaceutical form and in accordance with a conventional method, and an appropriate amount of the combination of one or more thereof may be contained. As these pharmacologically-active components or physiologically-active components, for example, effective ingredients in various medicines described in Approval Standards for Manufacture (Import) of Nonprescription Drugs, 2000 Edition (under the editorship of Yakuji Shinsa Kenkyukai) can be listed.

Specifically, examples of the pharmacologically-active components or the physiologically-active components include components as follows.

Antihistamine agents or anti-allergic agents: for example, ketotifen fumarate, chlorpheniramine maleate, diphenhydramine hydrochloride, sodium cromoglycate, tranilast, pemirolast potassium or the like.

Decongestants: for example, tetrahydrozoline hydrochloride, naphazoline hydrochloride, naphazoline sulfate, epinephrine hydrochloride, ephedrine hydrochloride, methylephedrine hydrochloride or the like.

Bactericides: for example, acrinol, cetylpyridinium, benzalkonium chloride, benzethonium chloride, chlorhexidine hydrochloride, chlorhexidine gluconate, alkyldiaminoethylglycine hydrochloride or the like.

Vitamins: for example, pyridoxine hydrochloride, flavin adenine dinucleotide sodium, cyanocobalamin, retinol acetate, retinol palmitate, panthenol, calcium pantothenate, tocopherol acetate or the like.

Amino acids: for example, potassium aspartate, magnesium aspartate, aminoethylsulfonic acid or the like.

Antiphlogistics: for example, dipotassium glycyrrhizinate, sodium azulene sulfonate, allantoin, ε-aminocaproic acid, berberine, lysozyme, pranoprofen or the like.

Astringents: for example, zinc flower, zinc lactate, zinc sulfate or the like.

Others: for example, sodium hyaluronate, sulfamethoxazole, sulfamethoxazole sodium, neostigmine methylsulfate or the like.

Moreover, in the ophthalmic aqueous composition of the present invention, within the range not impairing the effects of the present invention, various additives are appropriately selected based on its use and pharmaceutical formulation and in accordance with a conventional method, and an appropriate amount of the combination of one or more thereof may be contained. As these additives, for example, various excipients described in Japanese Pharmaceutical Excipients Directory 2007 (edited by the International Pharmaceutical Excipients Council Japan) can be listed. Examples of typical components include excipients as follows.

Thickeners: for example, carboxy vinyl polymer, hydroxyethyl cellulose, hydroxypropyl methylcellulose, methylcellulose, carboxymethylcellulose sodium, alginic acid, polyvinyl alcohol (fully or partially saponified material), polyvinylpyrrolidone, macrogol, sodium chondroitin sulfate, sodium hyaluronate or the like.

Sugars: for example, glucose, cyclodextrin or the like.

Sugar alcohols: for example, xylitol, sorbitol, mannitol or the like. These may be any of d-isomer, l-isomer, and dl-isomer.

Preservatives, bactericides, or antimicrobials: for example, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, Glokill (manufactured by Rhodia, trade name) or the like.

pH regulators: for example, hydrochloric acid, boric acid, aminoethylsulfonic acid, epsilon-aminocapronic acid, citric acid, acetic acid, sodium hydroxide, potassium hydroxide, calcium hydroxide, magnesium hydroxide, sodium hydrogen carbonate, sodium carbonate, borax, triethanolamine, diisopropanolamine, sulfuric acid, phosphoric acid, polyphosphoric acid, propionic acid, oxalic acid, gluconic acid, fumaric acid, lactic acid, tartaric acid, malic acid, succinic acid, gluconolactone, ammonium acetate or the like.

Stabilizers: for example, dibutylhydroxytoluene, tromethamol, sodium formaldehydesulfoxylate, tocopherol, sodium pyrosulfite, aluminum monostearate, glyceryl monostearate or the like.

Chelators: for example, ethylenediaminediacetic acid (EDDA), ethylenediaminetriacetic acid, ethylenediaminetetraacetic acid (edetic acid, EDTA), N-(2-hydroxyethyl)ethylenediaminetriacetic acid (HEDTA), diethylenetriaminepentaacetic acid (DTPA) or the like.

Preferable combinations of the (a) component, the (b) component (combination of two or three), and the (c) component in the ophthalmic aqueous composition of the present invention are shown below.

### Illustrative Example 1

Combination of sesame oil as the (a) component, Poloxamer 407 and polyoxyethylene hydrogenated castor oil 60 as the (b) component, and L-menthol as the (c) component.

### Illustrative Example 2

Combination of sesame oil as the (a) component, Poloxamer 407, polyoxyethylene hydrogenated castor oil 60, and polyoxyethylene castor oil 10 as the (b) component, and L-menthol as the (c) component.

### Illustrative Example 3

Combination of sesame oil as the (a) component, Poloxamer 407 and polyoxyethylene castor oil 35 as the (b) component, and L-menthol as the (c) component.

### Illustrative Example 4

Combination of sesame oil as the (a) component, Poloxamer 407, polyoxyethylene castor oil 35, and polyoxyethylene castor oil 10 as the (b) component, and L-menthol as the (c) component.

### Illustrative Example 5

Combination of sesame oil as the (a) component, Poloxamer 407, polyoxyethylene hydrogenated castor oil 60, and polyoxyethylene castor oil 35 as the (b) component, and L-menthol as the (c) component.

(6) Pharmaceutical Form

Specifically, examples of the pharmaceutical formulation of the ophthalmic aqueous composition of the present invention include eye drops (including eye drops that can be instilled in the eye in wearing contact lenses), eye washes (including eye washes that can wash the eye in wearing contact lenses), contact lens wetting solutions, contact lens care agents and the like. It is to be noted that the above-described contact lenses include every contact lenses such as hard contact lenses, oxygen-permeable hard contact lenses, soft contact lenses, and silicone hydrogel contact lenses.

As a packaging container of the ophthalmic aqueous composition of the present invention, a highly transparent plastic container is preferable. Examples of a material for such a plastic container include polycarbonate, polyethylene terephthalate, polyarylate, and polyethylene naphthalate. In particular, a container made of polyethylene terephthalate is preferable. In addition, it is preferable that a sterile container be used so as to use the ophthalmic aqueous composition safely.

Specific examples of packaged ophthalmic aqueous compositions include products in which eye drops (including eye drops that can be instilled in the eye in wearing contact lenses), eye washes (including eye washes that can wash the eye in wearing contact lenses), contact lens wetting solutions, contact lens care agents and the like are filled in containers having at least an optically transparent part.

The ophthalmic aqueous composition of the present invention is useful for a preventive or therapeutic agent for dry eye. Furthermore, the ophthalmic aqueous composition of the present invention can be used effectively as a cornea protective agent, and thus, is useful for eye disease prevention, and prevention or therapy for ophthalmia (snow blindness) and the like caused by ultraviolet light or the like. Furthermore, the ophthalmic aqueous composition of the present invention is also useful as a suppressing agent for inflammation caused by allergic substances. Furthermore, the ophthalmic aqueous composition of the present invention is useful for therapy for itchy eye, discomfort in wearing contact lenses, blurred vision and the like.

(7) Manufacturing Method

The ophthalmic aqueous composition of the present invention can be obtained by appropriately selecting a manufacturing method such that the average particle diameter of the emulsion is within a predetermined range. In the manufacturing method, examples of factors that influence the average particle diameter of the emulsion include the order of charging the components, the kind of an agitator, the agitating time, the agitating temperature and the like.

For example, after predetermined amounts of a vegetable oil, a non-ionic surfactant, a terpenoid, and other hydrophobic components are mixed with an agitator, purified water and other components are added thereto to be further mixed and the total volume is adjusted with purified water, and then, it is filtered by a 0.2 μm filter and is filled in a plastic container. Although a stirrer, a homo mixer, a propeller mixer, an agitating emulsifier, and a high pressure emulsifier can be used as the agitator, preferably, by performing agitation with the homo mixer or the agitating emulsifier, the emulsion having a predetermined average particle diameter is easily formed, and the effects of the present invention can be further improved.

Therefore, from another viewpoint, the present invention provides a manufacturing method of the ophthalmic aqueous composition having the effect of preventing or treating dry eye, or the effect of suppressing inflammation of the eye surface, which includes forming an oil-in-water emulsion having the average particle diameter of emulsion particles within the range of 30 nm to 300 nm by the addition of a vegetable oil, a non-ionic surfactant, and a terpenoid to a support containing water.

2. Method for Imparting Preventive or Therapeutic Effect on Dry Eye, Cornea Protective Effect, Effect of Preventing or Treating Allergic Symptoms of Eye Surface, Effect of Suppressing Inflammation of Eye Surface and the Like to Ophthalmic Aqueous Composition

As described above, according to the ophthalmic aqueous composition of the present invention, the average particle diameter of emulsion particles of the oil-in-water emulsion is made to be within a predetermined range by a combination of the (a) to (c) components so that the effect of stabilizing a tear film and a cornea protective effect are imparted to the ophthalmic aqueous composition, and thus, dryness of the eye is relieved and the effect of preventing or treating dry eye can be imparted to the ophthalmic aqueous composition. Furthermore, the present invention imparts a cornea protective effect to the ophthalmic aqueous composition, and thus, the effect of preventing or treating allergic symptoms of the eye surface caused by allergic substances and the effect of preventing or treating ophthalmia (snow blindness) and the like caused by ultraviolet light or the like can be imparted to the ophthalmic aqueous composition. Furthermore, according to the ophthalmic aqueous composition of the present invention, the effect of suppressing or relieving inflammation of the eye surface caused by common allergic substances can be imparted to the ophthalmic aqueous composition.

Therefore, from another viewpoint, the present invention provides a method for imparting the effect of stabilizing a tear film to the ophthalmic aqueous composition, a method for imparting a cornea protective effect to the ophthalmic aqueous composition, a method for imparting the effect of preventing or treating dry eye to the ophthalmic aqueous composition, a method for imparting the effect of preventing or treating allergic symptoms of the eye surface caused by allergic substances to the ophthalmic aqueous composition, a method for imparting the effect of preventing or treating ophthalmia (snow blindness) and the like caused by ultraviolet light or the like to the ophthalmic aqueous composition, or a method for imparting the effect of suppressing or relieving inflammation of the eye surface to the ophthalmic aqueous composition, which includes adjusting the average particle diameter of emulsion particles in an oil-in-water emulsion to the range of 30 nm to 300 nm by blending a vegetable oil, a non-ionic surfactant, and a terpenoid to the ophthalmic aqueous composition.

Furthermore, from another viewpoint, the present invention provides the use of a vegetable oil, a non-ionic surfactant, and a terpenoid for manufacturing the ophthalmic aqueous composition comprising an oil-in-water emulsion including a vegetable oil, a non-ionic surfactant, and a terpenoid, having an average particle diameter of emulsion particles within the range of 30 nm to 300 nm, and having the effect of preventing or treating dry eye, a cornea protective effect, the effect of preventing eye disease caused by infection or the like, the effect of preventing or treating ophthalmia (snow blindness) and the like caused by ultraviolet light or the like, or the effect of suppressing or relieving inflammation of the eye surface.

Furthermore, from another viewpoint, the present invention provides a composition comprising an oil-in-water emulsion including a vegetable oil, a non-ionic surfactant, and a terpenoid, and having an average particle diameter of emulsion particles within the range of 30 nm to 300 nm, for the use as the ophthalmic aqueous composition having the effect of preventing or treating dry eye, a cornea protective effect, the effect of preventing or treating allergic symptoms of the eye surface caused by allergic substances, the effect of preventing or treating ophthalmia (snow blindness) and the like caused by ultraviolet light or the like, or the effect of suppressing or relieving inflammation of the eye surface.

Furthermore, from another viewpoint, the present invention provides the use of a composition comprising an oil-in-water emulsion including a vegetable oil, a non-ionic surfactant, and a terpenoid, and having an average particle diameter of emulsion particles within the range of 30 nm to 300 nm, as the ophthalmic aqueous composition having the effect of preventing or treating dry eye, a cornea protective effect, the effect of preventing or treating allergic symptoms of the eye surface caused by allergic substances, the effect of preventing or treating ophthalmia (snow blindness) and the like caused by ultraviolet light or the like, or the effect of suppressing or relieving inflammation of the eye surface.

It is to be noted that, in the above-described respective methods, the kind and the content ratio of the respective components to be blended into the ophthalmic aqueous composition, the kind and the content ratio of other components to be blended, a pharmaceutical form of the composition and the like are the same as the ophthalmic aqueous composition of the present invention described above.

3. Method for Preventing or Treating Dry Eye, Cornea Protective Method, Method for Preventing or Treating Allergic Symptoms of Eye Surface, Method for Suppressing Inflammation of Eye Surface and the Like

In addition, as described above, the ophthalmic aqueous composition of the present invention is used as eye drops, eye washes or the like, and the composition is brought into contact with the cornea by methods such as being instilled in the eye and washing the eye so that the tear film can be stabilized, and further, the cornea can be protected. As a result, dryness of the eye is relieved and dry eye can be prevented or treated by the method. Furthermore, according to the ophthalmic aqueous composition of the present invention, with a cornea protective effect, allergic symptoms of the eye surface caused by allergic substances can be prevented or treated, and ophthalmia (snow blindness) and the like caused by ultraviolet light or the like can be prevented, suppressed or relieved. Furthermore, according to the ophthalmic aqueous composition of the present invention, inflammation of the eye surface caused by common allergic substances can be suppressed or relieved.

Therefore, from another viewpoint, the present invention further provides a method for stabilizing a tear film, a method for protecting a cornea, a method for preventing or treating dry eye, a method for preventing or treating allergic symptoms of the eye surface, a method for preventing or treating ophthalmia, or a method for suppressing or relieving inflammation of the eye surface, which includes bringing the ophthalmic aqueous composition comprising an oil-in-water emulsion including a vegetable oil, a non-ionic surfactant, and a terpenoid, and having an average particle diameter of emulsion particles within the range of 30 nm to 300 nm into contact with the cornea.

In these methods, the kind and the content ratio of the respective components to be blended into the ophthalmic aqueous composition, the kind and the content ratio of other components to be blended, a pharmaceutical form of the composition and the like are the same as the ophthalmic aqueous composition of the present invention described above.

### EXAMPLES

Hereinafter, Examples are given below to illustrate the present invention in detail; however the present invention is not limited to these Examples.

### Test Example 1

### Stability Test of Tear Film Breakup Time (NIBUT)

NIBUT is an abbreviation of non-invasive tear film breakup time, and among BUTs, it is BUT measured by a method that is closer to the natural state without putting a load such as a stain.

1-1. Preparation of Formulation:

In accordance with the following Table 1, eye drops of Examples 1 to 3 and Comparative Examples 1 to 4 were prepared by a combination of a homo mixer and an agitating emulsifier to obtain testing liquids. It is to be noted that, in the formulation in Table 1, products that meet The Japanese Pharmacopoeia Fifteenth Edition were used as sesame oil and Polysorbate 80, respectively, a product that meets Japanese Pharmaceutical Excipients 2003 was used as polyoxyethylene hydrogenated castor oil 60, and a product that meets Japanese Pharmaceutical Excipients 2003 was used as Poloxamer 407.

1-2. Measurement of Particle Diameter:

With respect to the respective testing liquids after the preparation, the average particle diameter of the emulsion was measured. The measurement of the average particle diameter was performed using a particle diameter measuring device (FPAR-1000 (Otsuka Electronics Co., Ltd.)) by dynamic light scattering (photon correlation method). Detailed measurement conditions are as follows. The measurement results are also described in Table 1.

Measurement Conditions
Temperature 25° C.
ND filter AUTO
Probe for concentrated solution
Angle 160°
Measurement time 180 seconds
Repeat count 1 time
Dust cut 10 times (upper 10%, lower 100%)

Adjustment of amount of light
homodyne amount of light optimum 30000 cps MAX 50000 cps MIN 10000 cps

Average particle diameter analytical technique
cumulant method analysis

Solvent condition
refractive index 1.3313

It is to be noted that, in the particle diameter measurement by dynamic light scattering, the viscosity of the testing liquid may influence the measurement result. Although it is considered that there is little influence in the vicinity of the viscosity of the testing liquid in the present test examples, in order to obtain more precise measurement result, a viscosity correction was performed just in case. Specifically, the viscosity of the testing liquid was measured using a tuning-fork viscometer (Viscometer SV-10 (A&D)), the viscosity measurement value was input in measuring the particle diameter, and the correction was applied in result analysis to obtain the result.

1-3. Measurement of NIBUT:

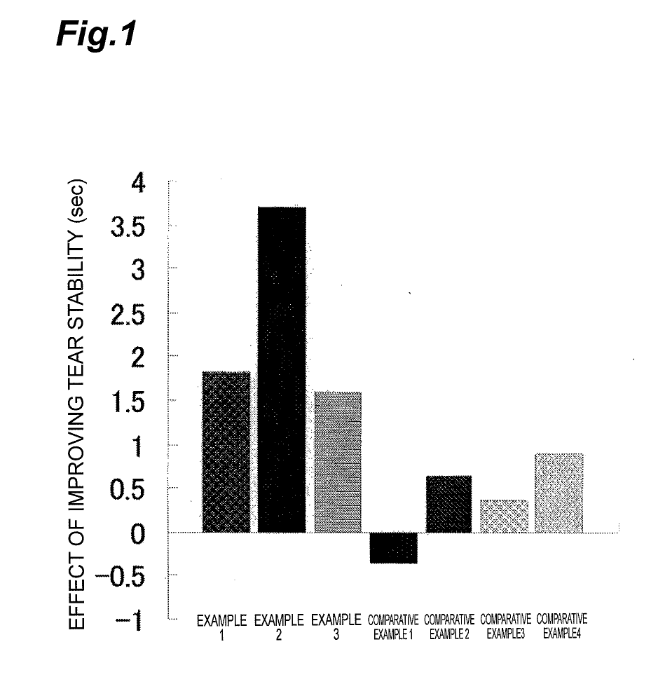

The measurement of NIBUT was performed by measuring time to tear breakup while keeping the eyelids open, using Interferometer DR-1 (Kowa Company, Ltd.). As test subjects of the present test, 28 eyes were selected among subjects having NIBUT of less than 10 seconds. Different two kinds of eye drops were selected at random from eight kinds of eye drops described in Table 1 and allocated to left and right eyes of the test subject, and three examples (that is, three eyes) were tested for each eye drop. It is to be noted that, as a method for being instilled in the eye, one drop was instilled one time for each ophthalmic drug. NIBUT was measured just after the instillation, and the effect of improving tear stability by the ophthalmic solution was evaluated based on the following equation. The result is shown in FIG. 1.

Effect of improving tear stability(sec)=(NIBUT after instillation)−(NIBUT before instillation)

TABLE 1
(Unit: w/v %)

| | | | | Comparative | Comparative | Comparative | Comparative |
|---|---|---|---|---|---|---|---|
| | Example 1 | Example 2 | Example 3 | Example 1 | Example 2 | Example 3 | Example 4 |
| Sodium chloride | 0.4 | 0.4 | 0.4 | 0.4 | 0.4 | 0.4 | 0.4 |
| Boric acid | 0.5 | 0.5 | 0.5 | 0.5 | 0.5 | 0.5 | 0.5 |
| Borax | 0.1 | 0.1 | 0.1 | 0.1 | 0.1 | 0.1 | 0.1 |
| L-menthol | 0.005 | 0.005 | 0.005 | — | 0.003 | 0.005 | 0.005 |
| Sesame oil | 0.05 | 0.05 | 0.05 | 0.05 | 0.025 | 0.1 | 0.05 |
| Poloxamer 407 | 0.1 | 0.1 | 0.1 | — | 0.1 | 0.1 | 0.1 |
| Polyoxyethylene | 0.2 | 0.25 | 0.5 | 0.25 | 0.5 | — | — |
| hydrogenated castor oil 60 | | | | | | | |
| Polysorbate 80 | — | — | — | — | — | 0.5 | 0.5 |
| 20% solution of | 0.0005 | 0.0005 | 0.0005 | 0.0005 | 0.0005 | 0.0005 | 0.0005 |
| polyhexanide hydrochloride | | | | | | | |
| Hydrochloric acid | qs | qs | qs | qs | qs | qs | qs |
| Sodium hydroxide | qs | qs | qs | qs | qs | qs | qs |
| Purified water | balance (97 mL | balance (97 mL | balance (97 mL | balance (97 mL | balance (97 mL | balance (97 mL | balance (97 mL |
| | or more) | or more) | or more) | or more) | or more) | or more) | or more) |
| Total | 100 mL | 100 mL | 100 mL | 100 mL | 100 mL | 100 mL | 100 mL |
| pH | 7.5 | 7.5 | 7.5 | 7.5 | 7.5 | 7.5 | 7.5 |
| Average particle diameter (nm) | 256.8 | 115.7 | 43.3 | 85.5 | 27.9 | 451.4 | 29.2 |

As is obvious from the above Table 1 and FIG. 1, by instilling the testing liquids of Examples 1 to 3, each of which contains a vegetable oil, a non-ionic surfactant, and a terpenoid and has the average particle diameter of emulsion particles within the range of 30 to 300 nm, the tear film breakup time (NIBUT) was prolonged and the effect of improving the tear stability was seen. On the other hand, in the case of instilling the testing liquids of Comparative Examples 2 to 4, each of which has the average particle diameter of emulsion particles out of the range of 30 to 300 nm, and the testing liquid of Comparative Example 1, which has the average particle diameter of emulsion particles within the range of 30 to 300 nm but does not contain a terpenoid, the effect of improving the tear stability was extremely low, or what is even worse, the tear stability was lowered.

### Test Example 2

### Evaluation Test of Corneal Epithelial Cell Protective Effect from Drying Load

2-1. Preparation of Formulation:

In accordance with the following Table 2, eye drops of Examples 4 to 9 and Comparative Examples 5 to 8 were prepared by a conventional method to obtain testing liquids. It is to be noted that, in the formulation in Table 2, products that meet The Japanese Pharmacopoeia Fifteenth Edition were used as sesame oil and Polysorbate 80, respectively, and products that meet Japanese Pharmaceutical Excipients 2003 were used as polyoxyethylene hydrogenated castor oil 60, polyoxyethylene castor oil 10, and Poloxamer 407.

2-2. Measurement of Particle Diameter:

The average particle diameter of the emulsion particles was measured in the same manner as Test Example 1. The measurement results are also described in Table 2.

TABLE 2
(Unit: w/v %)

| | | | | | | | Comparative | Comparative | Comparative | Comparative |
|---|---|---|---|---|---|---|---|---|---|---|
| | Example 4 | Example 5 | Example 6 | Example 7 | Example 8 | Example 9 | Example 5 | Example 6 | Example 7 | Example 8 |
| Sodium chloride | 0.4 | 0.4 | 0.4 | 0.4 | 0.4 | 0.4 | 0.4 | 0.4 | 0.4 | 0.4 |
| Boric acid | 0.5 | 0.5 | 0.5 | 0.5 | 0.5 | 0.5 | 0.5 | 0.5 | 0.5 | 0.5 |
| Borax | 0.1 | 0.1 | 0.1 | 0.1 | 0.1 | 0.1 | 0.1 | 0.1 | 0.1 | 0.1 |
| L-menthol | 0.005 | 0.005 | 0.001 | 0.05 | 0.0005 | 0.025 | 0.005 | — | 0.005 | 0.003 |
| Sesame oil | 0.05 | 0.05 | 0.01 | 0.01 | 0.005 | 0.005 | 0.05 | 0.05 | 0.1 | 0.025 |
| Poloxamer 407 | 0.1 | 0.1 | 0.1 | 0.1 | 0.05 | 0.05 | 0.1 | — | 0.1 | 0.1 |
| Polyoxyethylene | 0.5 | 0.15 | 0.05 | 0.1 | 0.025 | 0.05 | — | 0.25 | — | 0.5 |
| hydrogenated castor | | | | | | | | | | |
| oil 60 | | | | | | | | | | |
| Polysorbate 80 | — | — | — | — | — | — | 0.5 | — | 0.5 | — |
| Polyoxyethylene | — | — | 0.01 | 0.01 | 0.005 | 0.005 | — | — | — | — |
| castor oil 10 | | | | | | | | | | |
| Hydrochloric acid | qs | qs | qs | qs | qs | qs | qs | qs | qs | qs |
| Sodium hydroxide | qs | qs | qs | qs | qs | qs | qs | qs | qs | qs |
| Purified water | balance | balance | balance | balance | balance | balance | balance | balance | balance | balance |
| | (97 mL or | (97 mL or | (97 mL or | (97 mL or | (97 mL or | (97 mL or | (97 mL or | (97 mL or | (97 mL or | (97 mL or |
| | more) | more) | more) | more) | more) | more) | more) | more) | more) | more) |
| Total | 100 mL | 100 mL | 100 mL | 100 mL | 100 mL | 100 mL | 100 mL | 100 mL | 100 mL | 100 mL |
| pH | 7.5 | 7.5 | 7.5 | 7.5 | 7.5 | 7.5 | 7.5 | 7.5 | 7.5 | 7.5 |
| Average particle | 36.9 | 213.2 | 58.6 | 34.6 | 37.7 | 30.7 | 21.3 | 85.5 | 1000 or | 27.9 |
| diameter | | | | | | | | | more | |
| (nm) | | | | | | | | | | |

2-3. Evaluation of Corneal Epithelial Cell Protective Effect from Drying Load:

Cornel epithelial cell lines HCE-T (RIKEN BioResource Center) were seeded such that they are 1.0×10⁵ cell/mL per 1 well of a 96-well microtiter plate (Corning), and were cultivated for 72 hours under conditions of 37° C., 5% CO2, and a humidity of 90%. After confirming that the cells became confluent, the culture media were removed, and eye drops (testing liquids) described in Table 2 were added to the respective different wells at 50 microliters per 1 well and incubated for 1 minute at room temperature (sample treatment group). Furthermore, samples prepared by addition of cell culture media, instead of the eye drops, at 50 microliters per 1 well served as a control group (NT). After 1 minute, the respective testing liquids were completely removed from the sample treatment group (no treatment in NT), and a drying load was applied for 2 minutes using an air blower under air blowing conditions of a distance from the air blower of about 50 cm and 0.4 m/s.

After the drying addition, 100 μL of a detection reagent for viable cells Cell Titer-Glo (Promega) were added to each well, and an emission value produced by reaction with the viable cells was measured using Luminometer Glomax (Promega). Based on the measurement value, a cell viability of the sample treatment group with respect to the control group was calculated by using the following equation, and the cell protective effect by sample treatment from the drying load was evaluated.

cell viability(%)=100×(emission value of each sample treatment group)/(emission value of absorbance of control group)

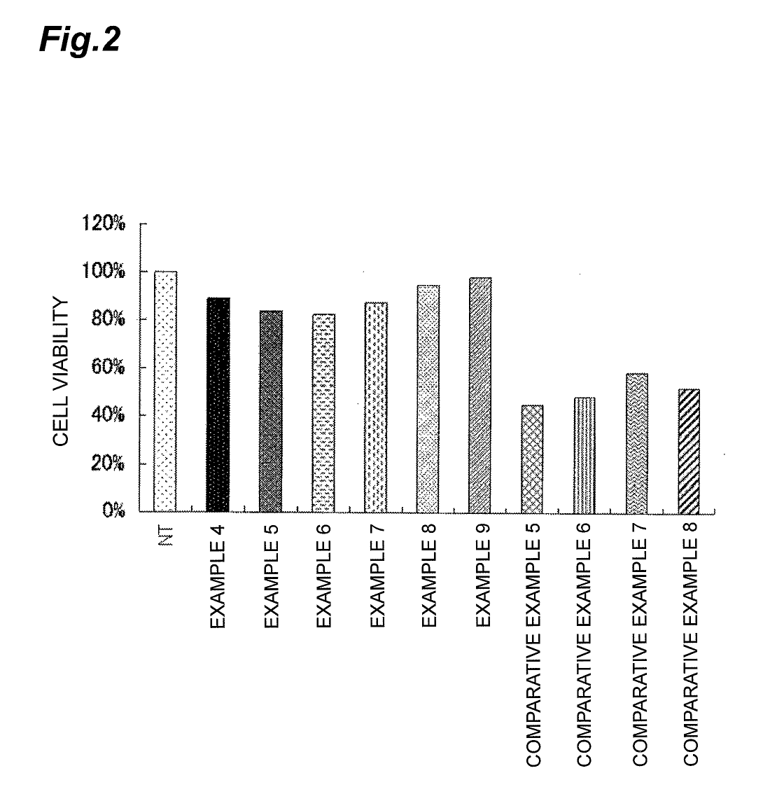

The above result is shown in FIG. 2. As is obvious from the above Table 2 and FIG. 2, it was confirmed that the cell treated with the testing liquids of Examples 4 to 9, each of which contains a vegetable oil, a non-ionic surfactant, and a terpenoid and has the average particle diameter of emulsion particles within the range of 30 to 300 nm, has a high cell viability and the testing liquids of Examples 4 to 9 excel in a cell protective effect against the drying load.

### Test Example 3

### Stability Test of Pharmaceutical

Among the testing liquids which were prepared and whose average particle diameters were measured in the above-described Test Examples 1 and 2, the respective testing liquids of Examples 2, 3, and 4 and the respective testing liquids of Comparative Examples 3 and 6 were filled in 15 mL PET eye drop containers to obtain eye drops. These eye drops were stored at 50° C. for a certain period, and a change in a property over time was visually evaluated. The result is shown in Table 3.

It is to be noted that the evaluation was performed based on the following criteria.

4 . . . The entire formulation is transparent and uniform.
3 . . . Transparency is extremely slightly reduced in the entire formulation, or about less than 10% of the total is separated.
2 . . . Transparency is slightly reduced in the entire formulation, or about 10 to 30% of the total is separated.
1 . . . Transparency is clearly reduced in the entire formulation, or 30% or more of the total is separated.

[Table 3]

TABLE 3
(Unit: w/v %)

| | | | | Com- | Com- |
|---|---|---|---|---|---|
| | | | | parative | parative |
| | Example 2 | Example 3 | Example 4 | Example 3 | Example 6 |
| Just after | 4 | 4 | 4 | 3 | 4 |
| manufacture | | | | | |
| 50° C. | 4 | 4 | 4 | 1 | 2 |
| 2 days | | | | | |
| 50° C. | 4 | 4 | 4 | 1 | 2 |
| 10 days | | | | | |

As is obvious from the above Table 3, the respective testing liquids of Examples 2, 3, and 4 do not have a noticeable change in a property after storing at 50° C. and are stable over time. In contrast, the testing liquid of Comparative Example 3, which has the particle diameter out of the range of the present invention, was unstable over time. Moreover, the testing liquid of Comparative Example 6, which has the average particle diameter of emulsion particles within the range of 30 to 300 nm but does not contain a terpenoid, resulted in inferior stability over time to Examples.

### Test Example 4

### Evaluation of Effect of Suppressing RANTES Production in Conjunctival Epithelial Cell

The effect of suppressing RANTES production, which is a cytokine having a migration enhancement effect, such as an acidocyte, was evaluated by the following method.

4-1. Preparation of Formulation:

In accordance with the following Table 4, eye drops of Example 10 and Comparative Example 9 were prepared by a conventional method to obtain testing liquids. It is to be noted that, in the formulation in Table 4, products that meet The Japanese Pharmacopoeia Fifteenth Edition were used as sesame oil and Polysorbate 80, respectively, and products that meet Japanese Pharmaceutical Excipients 2003 were used as polyoxyethylene hydrogenated castor oil 60 and Poloxamer 407.

4-2. Measurement of Particle Diameter:

The average particle diameter of the emulsion particles was measured in the same manner as Test Example 1. The measurement results are also described in Table 4.

4-3. Evaluation of Effect of Suppressing RANTES Production in Conjunctival Epithelial Cell:

Human conjunctival epithelial cell lines 1-5c-4 (ATCC) were seeded such that they are 1.0×10⁵ cells/200 μL per 1 well of a 96-well microtiter plate (Corning), and were cultivated for 24 hours under conditions of 37° C. and 5% CO₂ using Medium 199 (Invitrogen) to which 10% (v/v) Fetal bovine serum is added as culture media.

The culture media were removed by suction from wells of the 96-well microtiter plate, 60 μL of eye drops described in the following Table 4 and 140 μL of culture media were added, and cultivation was performed for 1 hour under conditions of 37° C. and 5% CO₂.

Subsequently, Recombinant Human TNF-α (R&D Systems) was prepared to a concentration of 1 μg/mL using a culture medium, 2 μL thereof was added to each well, and cultivation was performed for 24 hours under conditions of 37° C. and 5% CO₂. A cell culture supernatant was collected in a new 96-well microtiter plate and cryopreserved at −80° C. until the next operation.

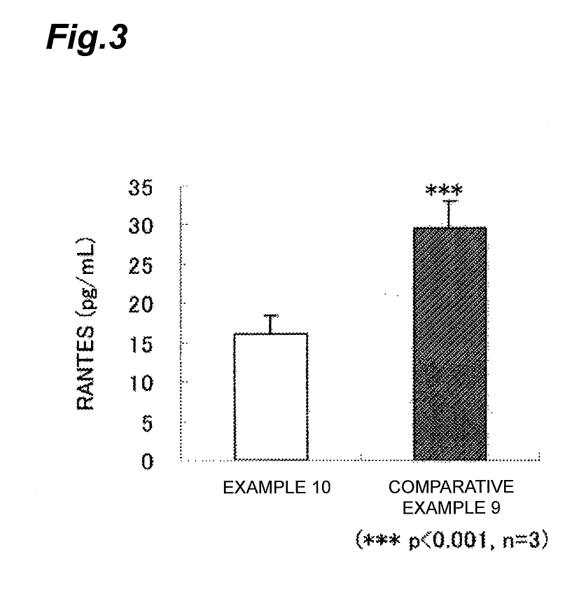

CCL5/RANTES Quantikine (R&D Systems) was prepared, the cryopreserved cell culture supernatant was thawed at room temperature, and a concentration of RANTES was quantitated by an ELISA method in accordance with the instructions attached to the kit. In this case, a microplate reader device (VersaMax manufactured by Molecular Devices, LLC) in which a measurement wavelength was set to 450 nm and a correction wavelength was set to 540 nm was used for the measurement of absorbance (temperature in device 20 to 25° C.). The result is shown in FIG. 3.

TABLE 4
(Unit: w/v %)

| Formulation | Example 10 | Comparative Example 9 |
|---|---|---|
| Sodium chloride | 0.4 | 0.4 |
| Boric acid | 0.5 | 0.5 |
| Borax | 0.1 | 0.1 |
| L-menthol | 0.03 | — |
| Sesame oil | 0.1 | — |
| Poloxamer 407 | 0.5 | — |
| Polyoxyethylene | 0.5 | 0.5 |
| hydrogenated castor oil 60 | | |
| Polysorbate 80 | — | — |
| Purified water | balance | balance |
| | (97 mL or more) | (97 mL or more) |
| Total | 100 mL | 100 mL |
| pH | 7.5 | 7.5 |
| Particle diameter | 54.2 | — |

As is obvious from the above Table 4 and FIG. 3, the cell treated with the testing liquid of Example 10, which contains a vegetable oil, a non-ionic surfactant, and a terpenoid and has the average particle diameter of emulsion particles within the range of 30 to 300 nm, exhibits a significant effect of suppressing RANTES production compared with the cell treated with the testing liquid of Comparative Example 9 (n=3, p<0.001, by Dunnett test). According to this result, the testing liquid of Example 10 is considered to have the effect of suppressing inflammation of the eye surface caused by allergic substances.

### Test Example 5

### Evaluation of Antioxidant Potential

5-1. Preparation of Formulation:

In accordance with the following Table 5, eye drops of Examples 10, 11 and Comparative Example 9 were prepared by a conventional method to obtain testing liquids. It is to be noted that, in the formulation in Table 5, a product that meets The Japanese Pharmacopoeia Fifteenth Edition was used as sesame oil, respectively, and products that meet Japanese Pharmaceutical Excipients 2003 were used as polyoxyethylene hydrogenated castor oil 60, and Poloxamer 407.

5-2. Measurement of Particle Diameter:

The average particle diameter of the emulsion particles was measured in the same manner as Test Example 1. The measurement results are also described in Table 5.

TABLE 5
(Unit: w/v %)

| | | | Comparative |
|---|---|---|---|
| Formulation | Example 10 | Example 11 | Example 9 |
| Sodium chloride | 0.4 | 0.4 | 0.4 |
| Boric acid | 0.5 | 0.5 | 0.5 |
| Borax | 0.1 | 0.1 | 0.1 |
| L-menthol | 0.03 | 0.03 | — |
| Sesame oil | 0.1 | 0.1 | — |
| Poloxamer 407 | 0.5 | — | — |
| Polyoxyethylene | 0.5 | 0.5 | 0.5 |
| hydrogenated castor oil 60 | | | |
| Purified water | Balance | Balance | Balance |
| | (97 mL or | (97 mL or | (97 mL or |
| | more) | more) | more) |
| Total | 100 mL | 100 mL | 100 mL |
| pH | 7.5 | 7.5 | 7.5 |
| Particle diameter | 54.2 | 71.3 | — |

5-3. Evaluation of Antioxidant Potential

By using PAO antioxidant potential measuring kit (Japan Institute for the Control of Aging, NIKKEN SEIL Co., Ltd), antioxidant potential of the testing liquids described in Table 5 was measured in accordance with the instructions attached to the kit and calculated as Cu reduction power. In this case, a microplate reader device (VersaMax manufactured by Molecular Devices, LLC) in which a measurement wavelength was set to 490 nm was used for the measurement of absorbance (temperature in device 20 to 25° C.).

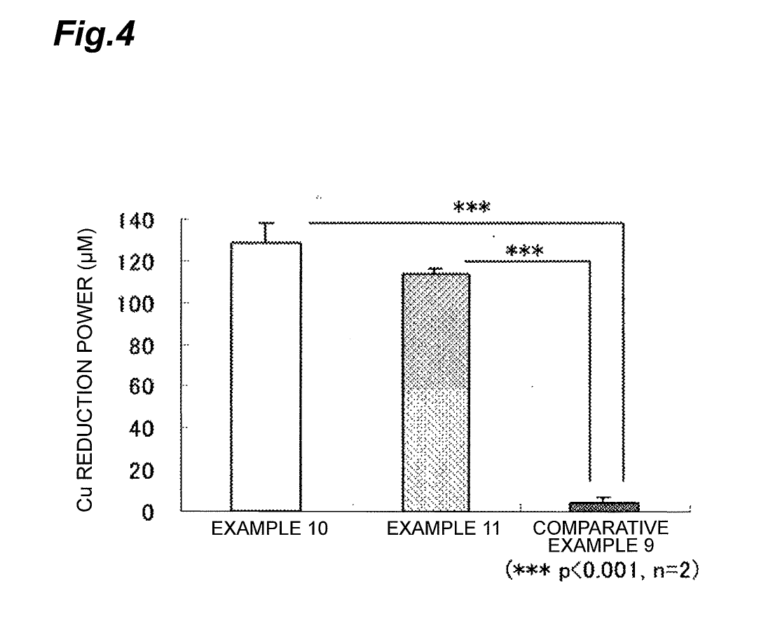

The result is shown in FIG. 4. As is obvious from the above Table 5 and FIG. 4, it could be confirmed that the testing liquids of Example 10 and Example 11, each of which contains a vegetable oil, a non-ionic surfactant, and a terpenoid and has the average particle diameter of emulsion particles within the range of 30 to 300 nm, have significantly high antioxidant potential compared with the testing liquid of Comparative Example 9 (n=2, p<0.001, by Tukey-kramer test). According to the comparison of the results of Example 10 and Example 11, the testing liquid of Example 10, which contains Poloxamer, tends to have higher antioxidant potential.

### Formulation Example

Ophthalmic aqueous compositions (formulation examples 1 to 10) were prepared by formulation described in the following Table 6. The measurement of the average particle diameter of the emulsion was performed in the same manner as Test Example 1. In the formulation in Table 6, products that meet The Japanese Pharmacopoeia Fifteenth Edition were used as sesame oil and Polysorbate 80, respectively, and products that meet Japanese Pharmaceutical Excipients 2003 were used as polyoxyethylene hydrogenated castor oil 60 and Poloxamer 407. Hydrochloric acid and sodium hydroxide were used for adjusting pH and added such that the ophthalmic aqueous composition has pH described in Table 6. Purified water was added such that the total volume of the ophthalmic aqueous composition is 100 mL.

TABLE 6
Unit in Table: w/v %

| | | | | | | | | | | Oph- |
|---|---|---|---|---|---|---|---|---|---|---|
| | Oph- | Oph- | Oph- | Oph- | Oph- | Oph- | Oph- | Oph- | Oph- | thalmic |
| | thalmic | thalmic | thalmic | thalmic | thalmic | thalmic | thalmic | thalmic | thalmic | solution |
| | solution | solution | solution | solution | solution | solution | solution | solution | solution | formu- |
| | formu- | formu- | formu- | formu- | formu- | formu- | formu- | formu- | formu- | lation |
| | lation | lation | lation | lation | lation | lation | lation | lation | lation | example |
| Unit: g/100 mL | example 1 | example 2 | example 3 | example 4 | example 5 | example 6 | example 7 | example 8 | example 9 | 10 |
| Aminoethylsulfonic acid | 0.100 | — | — | — | 0.100 | — | — | 0.500 | — | — |
| Sodium chondroitin sulfate | 0.500 | 0.500 | — | — | — | 0.500 | — | — | — | — |
| Polyvinylpyrrolidone K30 | — | — | 1.000 | — | — | — | — | 0.500 | — | — |
| Hydroxypropyl methylcellulose | — | — | — | 0.100 | — | — | — | — | — | — |
| Hydroxyethyl cellulose | — | — | — | — | 0.100 | — | 0.100 | — | — | — |
| Potassium chloride | 0.050 | — | 0.100 | 0.150 | 0.150 | 0.150 | — | 0.100 | — | — |
| Calcium chloride hydrate | — | — | 0.010 | 0.005 | 0.015 | 0.015 | 0.015 | 0.015 | — | — |
| Sodium chloride | 0.250 | 0.400 | 0.500 | 0.500 | 0.450 | 0.600 | — | — | 0.400 | 0.500 |
| Magnesium sulfate hydrate | — | 0.010 | — | — | — | 0.010 | — | 0.010 | — | — |
| Sodium hyaluronate | 0.004 | 0.100 | — | — | 0.001 | 0.050 | — | — | — | — |
| Sodium edetate | — | — | 0.001 | 0.050 | 0.100 | — | 0.025 | 0.100 | — | — |
| Boric acid | 1.000 | 1.200 | 1.000 | 0.500 | 0.500 | 0.500 | 1.500 | 1.000 | 0.500 | 0.500 |
| Borax | 0.100 | 0.100 | 0.050 | 0.100 | 0.100 | 0.100 | 0.200 | 0.200 | 0.100 | 0.100 |
| Poloxamer 407 | 0.100 | 0.100 | 0.100 | 0.100 | 0.100 | 0.100 | 0.100 | 0.100 | 0.100 | 0.100 |
| L-menthol | 0.005 | 0.005 | 0.005 | 0.005 | 0.005 | 0.005 | 0.005 | 0.005 | 0.005 | 0.005 |
| Sesame oil | 0.100 | 0.050 | 0.050 | 0.050 | 0.050 | 0.050 | 0.050 | 0.050 | 0.050 | 0.050 |
| Polyoxyethylene hydrogenated | 0.500 | — | 0.250 | 0.250 | 0.250 | 0.250 | 0.250 | — | 0.200 | 1.000 |
| castor oil 60 | | | | | | | | | | |
| Polysorbate 80 | — | 0.500 | — | — | — | — | — | 0.300 | — | — |
| 20% solution of polyhexamide | — | 0.0005 | — | 0.0003 | — | 0.0003 | — | 0.0003 | — | — |
| hydrochloride | | | | | | | | | | |
| Hydrochloric acid | qs | qs | qs | qs | qs | qs | qs | qs | qs | qs |
| Sodium hydroxide | qs | qs | qs | qs | qs | qs | qs | qs | qs | qs |
| Purified water | qs | qs | qs | qs | qs | qs | qs | qs | qs | qs |
| Total | 100 mL | 100 mL | 100 mL | 100 mL | 100 mL | 100 mL | 100 mL | 100 mL | 100 mL | 100 mL |
| pH | 7.0 | 6.5 | 6.5 | 7.5 | 7.5 | 7.5 | 6.5 | 7.5 | 7.5 | 7.5 |
| Average particle diameter | 213.3 | 181.4 | 136.1 | 168.6 | 86.7 | 210.3 | 99.2 | 275.3 | 207.1 | 38.8 |

1. An ophthalmic aqueous composition comprising an oil-in-water emulsion including a vegetable oil, a non-ionic surfactant, and a terpenoid, wherein an average particle diameter of emulsion particles is within the range of 30 nm to 300 nm.

2. The composition according to claim 1, wherein the vegetable oil is sesame oil.

3. The composition according to claim 1, wherein the non-ionic surfactant is at least one selected from the group consisting of a polyoxyethylene sorbitan fatty acid ester, polyoxyethylene hydrogenated castor oil, polyoxyethylene castor oil, and a polyoxyethylene-polyoxypropylene block copolymer.

4. The composition according to claim 1, wherein the non-ionic surfactant comprises a polyoxyethylene-polyoxypropylene block copolymer and another non-ionic surfactant.

5. The composition according to claim 1, wherein the terpenoid is at least one selected from the group consisting of menthol, menthone, camphor, borneol, and geraniol.

6. The composition according to claim 2, wherein the non-ionic surfactant is at least one selected from the group consisting of a polyoxyethylene sorbitan fatty acid ester, polyoxyethylene hydrogenated castor oil, polyoxyethylene castor oil, and a polyoxyethylene-polyoxypropylene block copolymer.

7. The composition according to claim 2, wherein the non-ionic surfactant comprises a polyoxyethylene-polyoxypropylene block copolymer and another non-ionic surfactant.

8. The composition according to claim 3, wherein the non-ionic surfactant comprises a polyoxyethylene-polyoxypropylene block copolymer and another non-ionic surfactant.

9. The composition according to claim 6, wherein the non-ionic surfactant comprises a polyoxyethylene-polyoxypropylene block copolymer and another non-ionic surfactant.

10. The composition according to claim 2, wherein the terpenoid is at least one selected from the group consisting of menthol, menthone, camphor, borneol, and geraniol.

11. The composition according to claim 3, wherein the terpenoid is at least one selected from the group consisting of menthol, menthone, camphor, borneol, and geraniol.

12. The composition according to claim 4, wherein the terpenoid is at least one selected from the group consisting of menthol, menthone, camphor, borneol, and geraniol.

13. The composition according to claim 6, wherein the terpenoid is at least one selected from the group consisting of menthol, menthone, camphor, borneol, and geraniol.

14. The composition according to claim 7, wherein the terpenoid is at least one selected from the group consisting of menthol, menthone, camphor, borneol, and geraniol.

15. The composition according to claim 8, wherein the terpenoid is at least one selected from the group consisting of menthol, menthone, camphor, borneol, and geraniol.

16. The composition according to claim 9, wherein the terpenoid is at least one selected from the group consisting of menthol, menthone, camphor, borneol, and geraniol.
